(19) United States
(12) Patent Application Publication
(10) Pub. No.: US 2025/0177268 A1
(43) Pub. Date: Jun. 5, 2025

(54) PROCESSO PARA A PRODUÇÃO DE ISOOLEATO DE ETILA E FÓRMULAS APLICADAS EM PRODUTOS COSMÉTICOS

(71) Applicant: BRASIL BIO FUELS S.A, SÃO JOÃO DA BALIZA (BR)

(72) Inventor: Setsuo Sato, Piracicaba (BR)

(21) Appl. No.: 18/596,168

(22) Filed: Mar. 5, 2024

(30) Foreign Application Priority Data
Nov. 30, 2023 (BR) 10 2023 025208 7

(51) Int. Cl. A61K 8/37; A61Q 5/12; A61Q 19/00; C07C 51/353; C07C 67/08

(52) U.S. Cl. CPC A61K 8/37; A61Q 5/12; A61Q 19/007; C07C 51/353; C07C 67/08

(57) ABSTRACT

The development of ethyl isooleate obtained from the isomerization of fatty acids from soy, palm, sunflower, rapeseed, peanut and its applications in the cosmetic field, more specifically, in formulations of face creams, body creams and hair conditioners is described. Isomerization creates non-linear branched chains that generate unique properties such as greater emollience, lubricity, solvency, spreadability, thermal and chemical stability, as well as greater fluidity in viscous systems. Ethyl isooleate is free from ocular and skin irritation, being completely non-allergenic. Ethyl oleate is an excellent solvent for active ingredients in ointments, creams, lotions. It works as an excellent excipient in delivery system formulations, helping the penetration of active ingredients into the skin. All of these characteristics above belong to ethyl oleate, however, from a physicochemical point of view, ethyl isooleate has superior properties to its isomer ethyl oleate.

The present invention is based on the development of ethyl isooleate obtained from the isomerization of fatty from soy, palm, sunflower, rapeseed, peanut and its applications in the cosmetic field, more specifically, in formulations of face creams, body creams and hair conditioners. Isomerization creates non-linear branched chains that generate unique properties such as greater emollience, lubricity, solvency, spreadability, thermal and chemical stability, as well as greater fluidity in viscous systems. Ethyl isooleate is free from ocular and skin irritation, being completely non-allergenic.

### RELATED CURRENT TECHNOLOGY

There are few applications for ethyl isooleate due to the fact that isooleic acid is still a co-product of the isomerization process of unsaturated fatty acids.

Technologies for its direct production using zeolite-based catalysts are being developed and in the future the production volumes of isooleic acid will increase due to its superior properties, compared to oleic acid.

Consequently, applications are concentrated on ethyl oleate that is used as a lubricating agent with the function of extending the life of car and truck engines.

Ethyl oleate has wide application in the food and cosmetics industries, it can also be used as a solvent in the pharmaceutical industry, as a lubricant, as a plasticizer, in the production of biodiesel or as an additive in diesel oil.

Ethyl oleate is an excellent solvent for active ingredients in ointments, creams, lotions. It works as an excellent excipient in delivery system formulations, helping the penetration of active ingredients into the skin.

The petroleum oil market in the United States was estimated at US$436.5 million per year in 2021, which corresponds to 25.86% of the global market which is equivalent to US$1.687 billion per year.

The most used raw material currently is palm oil due to the growth in its cultivation.

All of these characteristics above belong to ethyl oleate, however, from a physicochemical point of view, ethyl isooleate has superior properties to its isomer ethyl oleate.

### BRIEF DESCRIPTION OF THE FIGURE

FIG. 1 shows a schematic view of the production of ethyl isooleate.

### DESCRIPTION OF THE INVENTION

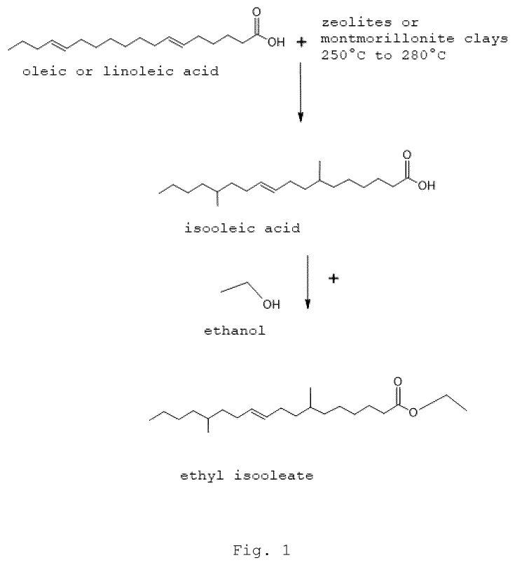

The production of ethyl isooleate shown in the diagram in FIG. 1 shows that it is the transformation of oleic acid into isooleate, through the isomerization process, where it is initially transformed into isooleic acid from the combination with zeolites, which acts as a catalyst at temperatures between 250° C. and 280° C.

In this initial step, oleic acid can be replaced by linoleic acid.

At this stage, zeolites can also be compensated by montmorrillonites in the same temperature variation.

The isooleic acid obtained is then combined with ethanol, resulting in ethyl isooleate.

The product obtained has been tested in accordance with the following cosmetic formulae, and complete application tests have been carried out to guarantee its safe use:

FACIAL MOISTURIZING CREAM A, with the following formula in table 1:

TABLE 1
FACIAL MOISTURIZING CREAM-A.

| | |
|---|---|
| Urea | 100 grams |
| Almond oil | 100 grams |
| Ethyl isooleate | 50 grams |
| Vitamin E 50% | 20 grams |
| Carbopol base cream | 730 grams |

FACIAL MOISTURIZING CREAM B, with the following formula in table 2:

TABLE 2
FACIAL MOISTURIZING CREAM-B.

| | |
|---|---|
| Collagen | 50 grams |
| Elastin | 30 grams |
| Ethyl isooleate | 100 grams |
| Vitamin E 50% | 20 grams |
| Vitamin A | 20 grams |
| Base cream | 780 grams |

BODY CREAM A, with the following formula in table 3:

TABLE 3
BODY CREAM A.

| | |
|---|---|
| Elastocell | 50 grams |
| D panthenol | 20 grams |
| Ethyl isooleate | 140 grams |
| Vitamin E 50% | 20 grams |
| Grape seed oil | 20 grams |
| Base cream | 750 grams |

BODY CREAM B, with the following formula in table 4:

TABLE 4
BODY CREAM-B.

| | |
|---|---|
| Elastin | 10 grams |
| Oil of Primula | 25 grams |
| Ethyl isooleate | 180 grams |
| Vitamin E 50% | 20 grams |
| Macadamia Oil | 80 grams |
| Cream base | 685 grams |

HAIR CONDITIONER A, with the following formula in table 5:

TABLE 5
HAIR CONDITIONER-A.

| | |
|---|---|
| Glycerin | 50 grams |
| Hydroxyethylcellulose | 60 grams |
| Ethoxylated fatty alcohol | 40 grams |
| Ethyl isooleate | 30 grams |
| Dietalonamine Palm Kernel Oil | 50 grams |
| Polysorbate | 40 grams |
| Sodium citrate | 10 grams |
| Perfume | 2 grams |
| Water | 718 grams |

HAIR CONDITIONER B, with the following formula in table 6:

TABLE 6
HAIR CONDITIONER-B

| | |
|---|---|
| Glycerin | 80 grams |
| Guar Gum | 60 grams |
| Polysorbate | 40 grams |
| Ethyl isooleate | 50 grams |
| Dietalonamine Palm Kernel | 40 grams |
| Oil | |
| ammonium quaternary | 40 grams |
| Sodium Gluconate | 5 grams |
| perfume | 2 grams |
| Glutaradehyde | 3 grams |
| Water | 680 grams |

1. Process for the production of ethyl isooleate characterized in that it is carried out by the transformation of oleic acid by isomerization using zeolites, at temperatures between 250° C. and 280° C., resulting in isooleic acid which is combined with ethanol.

2. Process for the production of ethyl isooleate, according to claim 1, characterized in that it is carried out by the transformation of oleic acid by isomerization using montmorrillonites, at a temperature between 250° C. and 280° C., resulting in isooleic acid which is combined with ethanol.

3. Process for the production of ethyl isooleate, according to claim 1, characterized in that it is carried out by the transformation of linoleic acid by isomerization using zeolites, at a temperature between 250° C. and 280° C., resulting in isooleic acid which is combined with ethanol.

4. Process for producing ethyl isooleate, according to claim 1, characterized in that it is carried out by transforming linoleic acid by isomerization using montmorillonite, at a temperature between 250° C. and 280° C., resulting in isooleic acid which is combined with ethanol.

5. Cosmetic using ethyl isooleate for use as a facial moisturizing cream characterized in that it consists of:

| | |
|---|---|
| Urea | 100 grams |
| Almond oil | 100 grams |
| Ethyl isooleate | 50 grams |
| Vitamin E 50% | 20 grams |
| Carbopol base cream | 730 grams |

6. Cosmetic using ethyl isooleate for use as a facial moisturizer, according to claim 5, characterized in that it consists of:

| | |
|---|---|
| Collagen | 50 grams |
| Elastin | 30 grams |
| Ethyl isooleate | 50 grams |
| Vitamin E 50% | 20 grams |
| Vitamin A | 20 grams |
| Base cream | 780 grams |

7. Cosmetic using ethyl isooleate for use as a body moisturizing cream characterized in that it consists of:

| | |
|---|---|
| Elastocell | 50 grams |
| D panthenol | 20 grams |
| Ethyl isooleate | 50 grams |
| Vitamin E 50% | 20 grams |
| Grape seed oil | 20 grams |
| Base cream | 750 grams |

8. Cosmetic using ethyl isooleate for use as a body moisturizer, according to claim 7, characterized in that it consists of:

| | |
|---|---|
| Elastin | 10 grams |
| Oil of Primula | 25 grams |
| Erhyl isooleate | 50 grams |
| Vitamin E 50% | 20 grams |
| Macadamia Oil | 80 grams |
| Base cream | 685 grams |

9. Cosmetic using ethyl isooleate for use as a hair conditioning cream characterized in that it consists of:

| | |
|---|---|
| Glycerin | 50 grams |
| Hydroxyethyl cellulose | 60 grams |
| Ethoxylated fatty alcohol | 40 grams |
| Ethyl isooleate | 50 grams |
| Dietalonamine Palm Kernel Oil | 50 grams |
| Polysorbate | 40 grams |
| Sodium citrate | 10 grams |
| Perfume | 2 grams |
| Water | 718 grams |

10. Cosmetic using ethyl isooleate for use as a hair conditioning cream, according to claim 9, characterized in that it consists of:

| | |
|---|---|
| Glycerin | 80 grams |
| Guar Gum | 60 grams |
| Polysorbate | 40 grams |
| Ethyl isooleate | 50 grams |
| Dietalonamine Palm Kernel Oil | 40 grams |
| Quaternary ammonium | 40 grams |
| Sodium gluconate | 5 grams |
| Perfume | 2 grams |
| Glutaradehyde | 3 grams |
| Water | 680 grams. |
